(19) United States
(12) Patent Application Publication
(10) Pub. No.: US 2014/0275215 A1
(43) Pub. Date: Sep. 18, 2014

(54) ANTI-CLUSTERIN MONOTHERAPY FOR CANCER TREATMENT

(71) Applicants: Shoshi Tessler, Zichron Ya'acov (IL); Joel Kaye, Netanya (IL); Tania Fine, Netanya (IL); Rina Kashi, Alfai-Menashe (IL)

(72) Inventors: Shoshi Tessler, Zichron Ya'acov (IL); Joel Kaye, Netanya (IL); Tania Fine, Netanya (IL); Rina Kashi, Alfai-Menashe (IL)

(73) Assignee: TEVA PHARMACEUTICAL INDUSTRIES, LTD., Petach-Tikva (IL)

(21) Appl. No.: 14/207,471

(22) Filed: Mar. 12, 2014

Related U.S. Application Data
(60) Provisional application No. 61/782,584, filed on Mar. 14, 2013.

(51) Int. Cl. C12N 15/113

(52) U.S. Cl. CPC C12N 15/113
USPC 514/44 A

(57) ABSTRACT

The present invention provides a method of treating cancer in a subject afflicted with cancer comprising administering to the subject an anti-clusterin oligonucleotide as a monotherapy to treat the cancer. The present invention also provides compositions for treating cancer in a subject afflicted with cancer, comprising an anti-clusterin oligonucleotide having the sequence CAGCAGCAGAGTCTTCATCAT (Seq. ID No.: 1). Additionally, the present invention provides pharmaceutical compositions for treating cancer in a subject afflicted with cancer, the composition comprising an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

This application claims the benefit of U.S. Provisional Application No. 61/782,584, filed Mar. 14, 2013, the contents of which is hereby incorporated by reference in its entirety.

Throughout this application, various publications are referenced, including referenced in parenthesis. Full citations for publications referenced in parenthesis may be found listed in alphabetical order at the end of the specification immediately preceding the claims. The disclosures of all referenced publications in their entireties are hereby incorporated by reference into this application in order to more fully describe the state of the art to which this invention pertains.

### REFERENCE TO SEQUENCE LISTING

This application incorporates-by-reference nucleotide and/or amino acid sequences which are present in the file named “140312_{—}2609_{—}85022_Sequence_Listing_ACK.txt,” which is 1 kilobyte in size, and which was created Mar. 11, 2014 in the IBM-PC machine format, having an operating system compatibility with MS-Windows, which is contained in the text file filed Mar. 12, 2014 as part of this application.

### BACKGROUND OF THE INVENTION

Clusterin is a secretable cytoprotective protein that is upregulated in response to a number of tumor cell killing interventions, specifically chemotherapy, hormone ablation therapy and radiation therapy.

Custirsen (also known as, TV-1011, OGX-011, and Custirsen sodium) is a second-generation antisense oligonucleotide (ASO) that inhibits clusterin expression. It has a 2″-MOE modification to the four ribonucleotides on both ends of the 21-mer phosphorothioate backbone. This results in an increased target binding affinity, resistance to degradation, and substantially better tissue PK than first-generation ASOs. The second-generation antisense molecules have a greater affinity for RNA targets and therefore greater potency, as demonstrated by the improved antisense potency observed in cell culture systems and in animals. In addition, the 2′-MOE modification results in decreased binding affinity to RNase H, the principal nuclease that cleaves ASO-bound RNA, which results in significantly improved tissue half-life in vivo (Gleave et al., 2002). This produces a longer duration of action, allowing less frequent dosing (Bennett et al., 2010). Finally, 2′-MOE ASOs have been reported to have a better safety profile than unmodified phosphorothioate ASOs (Henry et al., 2000).

Custirsen is designed specifically to bind to a portion of clusterin mRNA, resulting in the inhibition of the production of clusterin protein. The structure of custirsen is available, for example, in U.S. Pat. No. 6,900,187, the contents of which are incorporated herein by reference. A broad range of studies have shown that custirsen potently reduces the expression of clusterin, facilitates apoptosis, and sensitizes cancerous human prostate, breast, ovarian, lung, renal, bladder, and melanoma cells to chemotherapy (Miyake et al. 2005), see also, U.S. Patent Application Publication No. 2008/0119425 A1, the contents of which are incorporated herein by reference. Custirsen is not known to be effective for the treatment of cancer as a monotherapy.

New treatments for cancer are needed.

### SUMMARY OF THE INVENTION

The present invention provides a method of treating cancer in a subject afflicted with cancer comprising administering to the subject an anti-clusterin oligonucleotide as a monotherapy to treat the cancer.

The present invention provides a composition for treating cancer in a subject afflicted with cancer, comprising an anti-clusterin oligonucleotide having the sequence CAGCAGCAGAGTCTTCATCAT (Seq. ID No.: 1).

The present invention provides a composition for treating cancer in a subject afflicted with cancer, comprising an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

The present invention provides a pharmaceutical composition for treating cancer in a subject afflicted with cancer, the composition comprising an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

Aspects of the present invention relate to the use of a composition comprising an anti-clusterin oligonucleotide having the sequence CAGCAGCAGAGTCTTCATCAT (Seq. ID No.: 1) for treatment of cancer in a subject afflicted with cancer.

Aspects of the present invention relate to the use of a composition comprising an anti-clusterin oligonucleotide having the sequence CAGCAGCAGAGTCTTCATCAT (Seq. ID No.: 1) for preparation of a medicament for treatment of cancer in a subject afflicted with cancer.

The present invention provides a package for use in the treatment cancer in a subject afflicted with cancer, comprising an anti-clusterin oligonucleotide having the sequence CAGCAGCAGAGTCTTCATCAT (Seq. ID No.: 1).

### BRIEF DESCRIPTION OF THE DRAWINGS

FIG. 1: RPMI-8226 tumor volumes. Custirsen reduces tumor growth in vivo. All treatments stopped at day 52. Error Bars denote Standard Error.

FIG. 2: RPMI-8226 tumor volumes. Custirsen reduces tumor growth in vivo. Error Bars denote Standard Error.

FIG. 3: Effect of Custirsen against PC-3.

### DETAILED DESCRIPTION OF THE INVENTION

The present invention provides a method of treating cancer in a subject afflicted with cancer comprising administering to the subject an anti-clusterin oligonucleotide as a monotherapy to treat the cancer.

In some embodiments, the anti-clusterin oligonucleotide is administered to the subject periodically.

In some embodiments, the anti-clusterin oligonucleotide comprises nucleotides in the sequence CAGCAGCAGAGTCTTCATCAT (Seq. ID No.: 1).

In some embodiments, the anti-clusterin oligonucleotide is modified to increase its stability in vivo.

In some embodiments, the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

In some embodiments, the patient is afflicted with myeloma.

In some embodiments, the patient is afflicted with prostate cancer.

In some embodiments, the cancer is unresectable, advanced or metastatic cancer.

In some embodiments, the subject is a mammalian subject. In some embodiments, the mammalian subject is a human subject.

In some embodiments, the anti-clusterin oligonucleotide is administered to the subject intravenously in an aqueous solution comprising sodium ions.

In some embodiments, the anti-clusterin oligonucleotide is administered to the subject as 3 separate loading doses within a 5 to 9 day period at the beginning of treatment and then once weekly thereafter.

In some embodiments, the dose of the anti-clusterin oligonucleotide increases over each of the 3 loading doses.

In some embodiments, the first, second, and third loading doses are 320, 480, and 640 mg, respectively.

In some embodiments, 640 mg of the anti-clusterin oligonucleotide is administered to the human subject.

The present invention provides a composition for treating cancer in a subject afflicted with cancer, comprising an anti-clusterin oligonucleotide having the sequence CAGCAGCAGAGTCTTCATCAT (Seq. ID No.: 1).

The present invention provides a composition for treating cancer in a subject afflicted with cancer, comprising an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

The present invention provides a pharmaceutical composition for treating cancer in a subject afflicted with cancer, the composition comprising an anti-clusterin oligonucleotide having the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

Aspects of the present invention relate to the use of a composition comprising an anti-clusterin oligonucleotide having the sequence CAGCAGCAGAGTCTTCATCAT (Seq. ID No.: 1) for treatment of cancer in a subject afflicted with cancer.

Aspects of the present invention relate to the use of a composition comprising an anti-clusterin oligonucleotide having the sequence CAGCAGCAGAGTCTTCATCAT (Seq. ID No.: 1) for preparation of a medicament for treatment of cancer in a subject afflicted with cancer.

The present invention provides a package for use in the treatment cancer in a subject afflicted with cancer, comprising an anti-clusterin oligonucleotide having the sequence CAGCAGCAGAGTCTTCATCAT (Seq. ID No.: 1).

Anti-clusterin oligonucleotides may be used to treat many malignancies including prostate cancer, bladder cancer, ovarian cancer, renal cancer, melanoma, myeloma, breast cancer, lung cancer including NSCLC, and pancreatic cancer, in embodiments of the invention.

In some embodiments, an anti-clusterin oligonucleotide is administered as a monotherapy for treating myeloma or prostate cancer.

Each embodiment disclosed herein is contemplated as being applicable to each of the other disclosed embodiments. Thus, all combinations of the various elements described herein are within the scope of the invention.

It is understood that where a parameter range is provided, all integers within that range, and tenths thereof, are also provided by the invention. For example, “0.2-5 mg/kg/day” is a disclosure of 0.2 mg/kg/day, 0.3 mg/kg/day, 0.4 mg/kg/day, 0.5 mg/kg/day, 0.6 mg/kg/day etc. up to 5.0 mg/kg/day.

### TERMS

As used herein, and unless stated otherwise, each of the following terms shall have the definition set forth below.

As used herein, “about” in the context of a numerical value or range means±10% of the numerical value or range recited or claimed, unless the context requires a more limited range.

As used herein, “monotherapy” means a therapy that is administered to treat a disease, such as a cancer, without any other therapy that is used to treat the disease. A monotherapy for treating a cancer may optionally be combined with another treatment that is used to ameliorate a symptom of the cancer while not being directed against the cancer, but may not be combined with any other therapy directed against the cancer, such as a chemotherapeutic agent, hormone ablation therapy, or radiation therapy. Therefore, administering an anti-clusterin oligonucleotide as a monotherapy means administering the anti-clusterin oligonucleotide without radiation therapy, hormone ablation therapy, or any other chemotherapeutic agent. However, in some embodiments of the invention, agents that are not directed against the cancer, for example pain killers or corticosteroids, may be administered concurrently or simultaneously with the anti-clusterin oligonucleotide monotherapy.

As used herein, “anti-clusterin therapy” is therapy which reduces the expression of clusterin. An anti-clusterin therapy may be an anti-clusterin oligonucleotide.

Antisense oligonucleotides (ASOs) are stretches of single-strand deoxyribonucleic acid (DNA) complementary to messenger ribonucleic acid (mRNA) regions of a target gene. Because cellular ribosomal machinery translates mRNA into proteins, expression of specific proteins can be reduced by blocking or reducing this translation.

As used herein, “anti-clusterin oligonucleotide” refers to an antisense oligonucleotide which reduces clusterin expression, and comprises a nucleotide sequence that is complementary to clusterin-encoding mRNA. An example of an anti-clusterin oligonucleotide is custirsen.

As used herein, “custirsen” refers to an anti-clusterin oligonucleotide having nucleotides in the sequence CAGCAGCAGAGTCTTCATCAT (Seq. ID No.: 1), wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19. Custirsen can be in the form of Custirsen Sodium.

As used herein, “a human patient afflicted with” a condition, e.g. cancer, means a human patient who was been affirmatively diagnosed to have the condition.

As used herein, “effective” when referring to an amount of custirsen refers to the quantity of custirsen that is sufficient to yield a desired therapeutic response without undue adverse side effects (such as toxicity, irritation, or allergic response) commensurate with a reasonable benefit/risk ratio when used in the manner of this invention.

As used herein, “treating” encompasses, e.g., inhibition, regression, or stasis of the progression of cancer. Treating also encompasses the prevention or amelioration of any symptom or symptoms of cancer.

As used herein, “inhibition” of disease progression or disease complication in a subject means preventing or reducing the disease progression and/or a disease complication or symptom in the subject.

### Dosage Units

Administration of custirsen can be carried out using the various mechanisms known in the art, including naked administration and administration in pharmaceutically acceptable lipid carriers. For example, lipid carriers for antisense delivery are disclosed in U.S. Pat. Nos. 5,855,911 and 5,417,978, which are incorporated herein by reference. In general, custirsen is administered by intravenous (i.v.), intraperitoneal (i.p.), subcutaneous (s.c.), or oral routes, or direct local tumor injection. In some embodiments, custirsen is administered by i.v. injection.

The amount of anti-clusterin oligonucleotide administered may be from 40 to 640 mg, or from 300 to 640 mg. Administration of custirsen may be once in a seven day period, 3 times a week, or more specifically on days 1, 3 and 5, or 3, 5 and 7 of a seven day period. In some embodiments, administration of the antisense oligonucleotide is less frequent than once in a seven day period. In some embodiments, administration of the antisense oligonucleotide is more frequent than once in a seven day period. Dosages may be calculated by patient weight, and therefore in some embodiments a dose range of about 1-20 mg/kg, or about 2-10 mg/kg, or about 3-7 mg/kg, or about 3-4 mg/kg could be used. This dosage is repeated at intervals as needed. One clinical concept is dosing once per week with 3 loading doses during week one of treatment. In some embodiments, the dose of anti-clusterin oligonucleotide increases over the 3 loading doses. In some embodiments, the first, second, and third loading doses are 320, 480, and 640 mg, respectively. The amount of anti-clusterin oligonucleotide administered is one that has been demonstrated to be effective in human patients to inhibit the expression of clusterin in cancer cells.

A dosage unit may comprise a single compound or mixtures of compounds thereof. A dosage unit can be prepared for oral, injection, or inhalation dosage forms.

In some embodiments, custirsen may be formulated at a concentration of 20 mg/mL as an isotonic, phosphate-buffered saline solution for IV administration. In some embodiments, a formulation of custirsen may comprise 1, 2, 3, 4, 5, 6, 7, 8, 9, or 10% dextrose. In some embodiments, the formulation of custirsen may comprise 5% dextrose. In some embodiments custirsen may be supplied as a 32 mL solution containing 640 mg custirsen sodium in a single vial, or may be supplied as an 8 mL solution containing 160 mg custirsen sodium in a single vial. The drug product and active ingredient of custirsen sodium is a second-generation, 4-13-4 MOE-gapmer antisense oligonucleotide (ASO).

In some embodiments, custirsen may be added to 250 mL 0.9% sodium chloride (normal saline). In some embodiments, the dose may be administered using either a peripheral or central indwelling catheter intravenously as an infusion over 2 hours. Additionally, in some embodiments an infusion pump may be used.

General techniques and compositions for making dosage forms useful in the present invention are described in the following references: 7 Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol. 40 (Gilbert S. Banker, Christopher T. Rhodes, Eds.). These references in their entireties are hereby incorporated by reference into this application.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

### In Vivo Activity of Custirsen Against RPMI 8226 (Human Myeloma) Xenograft Model, Implanted Subcutaneously into Athymic Nude Mice

### Summary

This study aimed to evaluate the effect of custirsen (OGX-11, TV-1011) against human myeloma model in nude mice. The cells were implanted subcutaneously into female SCID mice with 7×10⁶ cells inoculum. On day 20, when the tumors reached 120-170 mm³, mice were sorted into two treatment groups (n=10): control group; and custirsen 40 mg/kg ip qd*5, then twk.

Tumors and body weights were measured weekly until termination of the study on day 71. Response to treatment was evaluated for tumor growth inhibition (TGI) and tumor growth delay (TGD).

Treatment with custirsen at a dose of 40 mg/kg was stopped after 7 injections due to toxicity. Nevertheless, as a monotherapy it significantly inhibited tumor growth by the end of the study.

### INTRODUCTION

The objective of this study was to evaluate the effect of custirsen (OGX-11, Tv-1011) against human myeloma model in nude mice.

### Materials and Methods

### Materials

RPMI 8226 (Human Plasmacytoma, Myeloma B Cells) ATCC # CCL-155;
RPMI medium (Beit Haemek);
ECM Gel (Mtrigel), Sigma-Aldrich, Cat # E1270, 5 ml;
HBSS (Beit Haemek);
Custirsen (OGX-11, TV-1011) 20 mg/ml, K-46138;
Sodium chloride, TEVA.

### Animals

70 CB.17 SCID female mice, 4-6 weeks old, 16-20 grams, obtained from Harlan animal breeding center.

### Cell Preparation

Harvested 14 flasks (T-175), passage 5, which were split 1:4. Sample from the cell suspension was taken for counting (0.3 ml in duplicates for CEDEX) before spun down. Cell viability was 85.6% and live cell concentration was 167×10⁵ cells/ml. The pellet was resuspended in HBSS to a final volume of 8 ml.

### Study Design

Tumors were implanted subcutaneously with RPMI-8226 cells into the right flank of the mouse on Day 0. Each animal received a s.c. injection 7×10 cells in 0.1 ml suspension. On day 20, mice were sorted by the optimal tumor volume (120-170 mm³) and were allocated into 5 groups of 10 mice. Mice were individually tagged and their tumor volume and body weight were monitored weekly during the study. Tumor size was measured by caliper and calculated using the formula:

$$π \times {( \frac{width}{2} )}^{2} \times length .$$

The treatment started on day 11 (0.2 ml per 20 grams v/w) and continued till day 52, after which the remaining mice were left for observation until day 71. Treatment regimen and doses are indicated in Table 1.

TABLE 1
Experimental design

Drug and treatment

| Gr. | N | Agent | mg/kg | Route | Schedule |
|---|---|---|---|---|---|
| 1 | 10 | Vehicle | — | ip | ip qd*5 + twk |
| 2 | 10 | Custirsen | 40 | ip | ip qd*5 + twk |

i.v.—intravenously, ip—intraperitoneally, twk—three times a week

### Results

The treatment responses for Day 51 are summarized in Table 3 and presented in FIG. 1. Treatment with custirsen at a dose of 40 mg/kg was stopped after 7 injections (5 consecutive+2 on the next week). On day 34, one mouse in custirsen 40 mg/kg group died. On Day 51, animals started to exit the study due to tumor size. Treatment with custirsen as a monotherapy inhibited tumor growth compared to control group. Custirsen alone inhibited tumor growth by 46% (p<0.05). The study was terminated on day 71, when 1-3 mice left in each group. Survival data is shown in Table 3.

TABLE 2
Summary of the results (day 51)

| | | | Δ mean | | | | Max BW | | |
|---|---|---|---|---|---|---|---|---|---|
| | Dose, | | tumor volume, | % TGI | No. of | No. of | (mean) | No. of | No. og |
| Compound | mg/kg | Regimen | (mean ± se) | day 51 | PR | CR | reduction | TRD | nTRD |
| 1. Vehicle | | i.p. qd*5, | 1228 ± 142 | | 0 | 0 | −1.3% | 0 | 0 |
| | | twk | | | | | day 34 | | |
| 2. Custirsen | 40 | ip qd*5, | 667 ± 87 | 46* | 0 | 0 | −4.7% | 1 | 0 |
| | | twk | | | | | day 34 | | |

PR—partial response: tumor reduction below baseline measurement;
CR—complete response: elimination of the tumor;
TRD—treatment related death;
NTRD—non treatment related death.

TABLE 3
Raw Data for Reduction in Myeloma Tumor Size with Custirsen Monotherapy

| | | | | | | | | % of | Δ | | | | | % of | Δ |
|---|---|---|---|---|---|---|---|---|---|---|---|---|---|---|---|
| | | Day 20, | | Day 27, | | Δ | Δ | base- | vol- | Day 34, | | Δ | Δ | base- | vol- |
| | | weight | volume | weight | volume | weight | % | line | ume | weight | volume | weight | % | line | ume |
| Vehicle | 1 | 16.4 | 119 | 16.7 | 188 | 0.3 | 2.1 | 102.1 | 69.9 | 16.8 | 292 | 0.4 | 2.6 | 102.6 | 173.4 |
| i.p. qd*5, | 2 | 20.0 | 125 | 21.1 | 208 | 1.1 | 5.4 | 105.4 | 83.7 | 20.2 | 274 | 0.2 | 0.7 | 100.7 | 149.4 |
| twk | 3 | 21.4 | 133 | 22.1 | 205 | 0.7 | 3.1 | 103.1 | 72.6 | 21.1 | 400 | −0.3 | −1.5 | 98.5 | 267.4 |
| | 4 | 22.7 | 137 | 22.0 | 209 | −0.7 | −2.9 | 97.1 | 72.0 | 21.1 | 332 | −1.6 | −6.8 | 93.2 | 195.4 |
| | 5 | 16.9 | 139 | 16.7 | 258 | −0.2 | −1.2 | 98.8 | 118.7 | 16.6 | 478 | −0.3 | −1.6 | 98.4 | 339.3 |
| | 6 | 21.2 | 144 | 21.1 | 215 | −0.1 | −0.6 | 99.4 | 71.1 | 20.4 | 294 | −0.8 | −4.0 | 96.0 | 149.4 |
| | 7 | 21.6 | 152 | 21.9 | 203 | 0.3 | 1.4 | 101.4 | 51.6 | 21.5 | 275 | 0.0 | −0.1 | 99.9 | 122.8 |
| | 8 | 21.1 | 159 | 21.4 | 210 | 0.3 | 1.6 | 101.6 | 51.2 | 20.7 | 351 | −0.3 | −1.5 | 98.5 | 192.6 |
| | 9 | 19.1 | 162 | 19.5 | 218 | 0.5 | 2.5 | 102.5 | 56.8 | 19.0 | 359 | −0.1 | −0.4 | 99.6 | 197.7 |
| | 10 | 20.8 | 163 | 20.4 | 246 | −0.4 | −2.0 | 98.0 | 83.7 | 20.6 | 446 | −0.1 | −0.7 | 99.3 | 282.9 |
| | Mean | 20.1 | 143.1 | 20.3 | 216.2 | 0.2 | 0.9 | 100.9 | 73.1 | 19.8 | 350.1 | −0.3 | −1.3 | 98.7 | 207 |
| | n | 10 | 10 | 10 | 10 | 10 | 10 | 10 | 10 | 10 | 10 | 10 | 10 | 10 | 10 |
| | SD | 2.1 | 15.5 | 2.0 | 20.7 | 0.5 | 2.6 | 2.6 | 19.8 | 1.8 | 71.9 | 0.6 | 2.6 | 2.6 | 68.5 |
| | SE | 0.7 | 4.9 | 0.6 | 6.5 | 0.2 | 0.8 | 0.8 | 6.3 | 0.6 | 22.7 | 0.2 | 0.8 | 0.8 | 21.7 |
| Curtirsen40 | 1 | 16.1 | 119 | 18.2 | 96 | 2.1 | 13.2 | 113.2 | −23.1 | 13.5 | 98 | −2.5 | −15.7 | 84.3 | −20.6 |
| ip qd*5, | 2 | 17.7 | 124 | 18.2 | 207 | 0.4 | 2.5 | 102.5 | 84.0 | 17.4 | 244 | −0.3 | −1.9 | 98.1 | 120.3 |
| twk | 3 | 17.1 | 125 | 18.0 | 171 | 0.8 | 4.8 | 104.8 | 46.2 | 17.1 | 223 | −0.1 | −0.4 | 99.6 | 97.9 |
| | 4 | 20.6 | 139 | 20.8 | 249 | 0.2 | 1.1 | 101.1 | 110.4 | 19.5 | 257 | −1.1 | −5.5 | 94.5 | 117.8 |
| | 5 | 19.7 | 141 | 19.7 | 222 | 0.1 | 0.3 | 100.3 | 81.2 | 19.1 | 305 | −0.5 | −2.7 | 97.3 | 163.9 |
| | 6 | 18.6 | 145 | 18.4 | 246 | −0.3 | −1.3 | 98.7 | 100.3 | 18.0 | 336 | −0.6 | −3.5 | 96.5 | 190.8 |
| | 7 | 18.4 | 151 | 18.9 | 319 | 0.4 | 2.4 | 102.4 | 168.8 | 18.2 | 371 | −0.2 | −1.1 | 98.9 | 219.9 |
| | 8 | 20.0 | 155 | 20.3 | 193 | 0.3 | 1.7 | 101.7 | 38.8 | 19.0 | 220 | −0.9 | −4.7 | 95.3 | 65.4 |
| | 9 | 18.4 | 162 | 18.6 | 217 | 0.2 | 0.8 | 100.8 | 55.3 | 17.2 | 266 | −1.2 | −6.7 | 93.3 | 104.3 |
| | 10 | 18.9 | 169 | 19.0 | 271 | 0.1 | 0.5 | 100.5 | 102.6 | 18.0 | 312 | −0.9 | −4.6 | 95.4 | 142.9 |
| | Mean | 18.5 | 142.8 | 19.0 | 219.2 | 0.4 | 2.6 | 102.6 | 76.4 | 17.7 | 263.1 | −0.8 | −4.7 | 95.3 | 120.3 |
| | n | 10 | 10 | 10 | 10 | 10 | 10 | 10 | 10 | 10 | 10 | 10 | 10 | 10 | 10 |
| | SD | 1.4 | 16.8 | 1.0 | 60.3 | 0.7 | 4.1 | 4.1 | 51.3 | 1.7 | 76.1 | 0.7 | 4.4 | 4.4 | 67.4 |
| | SE | 0.4 | 5.3 | 0.3 | 19.1 | 0.2 | 1.3 | 1.3 | 16.2 | 0.5 | 24.1 | 0.2 | 1.4 | 1.4 | 21.3 |

| | | | | | | % of | Δ | | | | | % of | Δ |
|---|---|---|---|---|---|---|---|---|---|---|---|---|---|
| | | Day 41, | | Δ | Δ | base- | vol- | Day 45, | | Δ | Δ | base- | vol- |
| | | weight | volume | weight | % | line | ume | weight | volume | weight | % | line | ume |
| Vehicle | 1 | 17.2 | 370 | 0.8 | 4.6 | 104.6 | 251.4 | 17.7 | 371 | 1.4 | 8.2 | 108.2 | 252.2 |
| i.p. qd*5, | 2 | 20.8 | 554 | 0.8 | 3.8 | 103.8 | 429.6 | 21.2 | 835 | 1.1 | 5.7 | 105.7 | 710.8 |
| twk | 3 | 21.6 | 688 | 0.2 | 1.1 | 101.1 | 555.7 | 22.2 | 945 | 0.8 | 3.7 | 103.7 | 812.5 |
| | 4 | 22.5 | 659 | −0.2 | −0.7 | 99.3 | 522.1 | 22.6 | 880 | 0.0 | −0.2 | 99.8 | 742.9 |
| | 5 | 17.3 | 816 | 0.4 | 2.6 | 102.6 | 677.2 | 17.8 | 1118 | 0.9 | 5.6 | 105.6 | 979.0 |
| | 6 | 21.0 | 577 | −0.2 | −0.9 | 99.1 | 432.6 | 21.7 | 811 | 0.4 | 2.1 | 102.1 | 666.8 |
| | 7 | 22.2 | 402 | 0.6 | 2.9 | 102.9 | 250.2 | 22.9 | 475 | 1.3 | 6.1 | 106.1 | 323.2 |
| | 8 | 21.3 | 505 | 0.2 | 1.0 | 101.0 | 346.5 | 21.8 | 795 | 0.7 | 3.5 | 103.5 | 636.2 |
| | 9 | 19.4 | 735 | 0.4 | 2.0 | 102.0 | 573.1 | 19.9 | 844 | 0.8 | 4.3 | 104.3 | 682.6 |
| | 10 | 21.2 | 977 | 0.4 | 2.0 | 102.0 | 814.1 | 22.7 | 1341 | 1.9 | 9.3 | 109.3 | 1178.6 |
| | Mean | 20.4 | 628.3 | 0.3 | 1.8 | 101.8 | 485 | 21.0 | 841.6 | 0.9 | 4.8 | 104.8 | 698 |
| | n | 10 | 10 | 10 | 10 | 10 | 10 | 10 | 10 | 10 | 10 | 10 | 10 |
| | SD | 1.9 | 186.8 | 0.3 | 1.8 | 1.8 | 180.8 | 1.9 | 278.4 | 0.5 | 2.8 | 2.8 | 272.7 |
| | SE | 0.6 | 59.1 | 0.1 | 0.6 | 0.6 | 57.2 | 0.6 | 88.0 | 0.2 | 0.9 | 0.9 | 86.2 |
| Curtirsen40 | 1 | | | | | | | | | | | | |
| ip qd*5, | 2 | 18.8 | 410 | 1.1 | 6.0 | 106.0 | 286.2 | 19.2 | 511 | 1.4 | 8.1 | 108.1 | 388.0 |
| twk | 3 | 17.9 | 295 | 0.8 | 4.4 | 104.4 | 170.4 | 18.8 | 272 | 1.7 | 9.8 | 109.8 | 147.2 |
| | 4 | 20.9 | 322 | 0.3 | 1.6 | 101.6 | 183.5 | 21.9 | 579 | 1.3 | 6.1 | 106.1 | 440.5 |
| | 5 | 19.8 | 454 | 0.1 | 0.4 | 100.4 | 313.0 | 20.5 | 587 | 0.8 | 4.1 | 104.1 | 446.1 |
| | 6 | 18.7 | 474 | 0.1 | 0.3 | 100.3 | 328.4 | 19.1 | 584 | 0.5 | 2.7 | 102.7 | 439.0 |
| | 7 | 19.5 | 560 | 1.1 | 5.9 | 105.9 | 409.6 | 20.6 | 826 | 2.1 | 11.4 | 111.4 | 675.2 |
| | 8 | 19.9 | 267 | −0.1 | −0.5 | 99.5 | 112.3 | 21.1 | 467 | 1.1 | 5.6 | 105.6 | 313.0 |
| | 9 | 17.6 | 320 | −0.8 | −4.2 | 95.8 | 157.8 | 18.7 | 428 | 0.3 | 1.5 | 101.5 | 266.0 |
| | 10 | 18.9 | 457 | 0.0 | 0.1 | 100.1 | 288.2 | 19.1 | 490 | 0.2 | 1.3 | 101.3 | 321.7 |
| | Mean | 19.1 | 395.4 | 0.3 | 1.5 | 101.5 | 249.9 | 19.9 | 527.3 | 1.0 | 5.6 | 105.6 | 381.9 |
| | n | 9 | 9 | 9 | 9 | 9 | 9 | 9 | 9 | 9 | 9 | 9 | 9 |
| | SD | 1.0 | 99.0 | 0.6 | 3.3 | 3.3 | 97.8 | 1.1 | 149.6 | 0.6 | 3.6 | 3.6 | 147.4 |
| | SE | 0.3 | 33.0 | 0.2 | 1.1 | 1.1 | 32.6 | 0.4 | 49.9 | 0.2 | 1.2 | 1.2 | 49.1 |

| | | | | | | % of | Δ | | | | | % of | Δ |
|---|---|---|---|---|---|---|---|---|---|---|---|---|---|
| | | Day 48, | | Δ | Δ | base- | vol- | Day 51, | | Δ | Δ | base- | vol- |
| | | weight | volume | weight | % | line | ume | weight | volume | weight | % | line | ume |
| Vehicle | 1 | 16.6 | 510 | 0.2 | 1.2 | 101.2 | 391.5 | 16.6 | 538 | 0.2 | 1.5 | 101.5 | 419.2 |
| i.p. qd*5, | 2 | 20.8 | 1000 | 0.7 | 3.5 | 103.5 | 875.0 | 21.5 | 1330 | 1.4 | 7.1 | 107.1 | 1205.0 |
| twk | 3 | 22.0 | 1111 | 0.6 | 3.0 | 103.0 | 978.8 | 22.0 | 1487 | 0.6 | 2.9 | 102.9 | 1354.7 |
| | 4 | 22.5 | 1140 | −0.1 | −0.5 | 99.5 | 1003.3 | 22.8 | 1408 | 0.2 | 0.8 | 100.8 | 1270.6 |
| | 5 | 17.4 | 1409 | 0.6 | 3.4 | 103.4 | 1269.6 | 18.3 | 2054 | 1.4 | 8.2 | 108.2 | 1915.0 |
| | 6 | 21.1 | 1002 | −0.1 | −0.7 | 99.3 | 857.5 | 21.4 | 1241 | 0.2 | 0.8 | 100.8 | 1096.6 |
| | 7 | 22.5 | 720 | 1.0 | 4.5 | 104.5 | 567.9 | 22.6 | 794 | 1.0 | 4.8 | 104.8 | 642.1 |
| | 8 | 21.5 | 986 | 0.4 | 2.0 | 102.0 | 827.1 | 21.7 | 1447 | 0.7 | 3.1 | 103.1 | 1289.0 |
| | 9 | 20.1 | 1125 | 1.0 | 5.4 | 105.4 | 963.5 | 20.1 | 1482 | 1.0 | 5.5 | 105.5 | 1320.2 |
| | 10 | 22.5 | 1388 | 1.7 | 8.4 | 108.4 | 1225.2 | 23.3 | 1934 | 2.6 | 12.4 | 112.4 | 1771.2 |
| | Mean | 20.7 | 1039.0 | 0.6 | 3.0 | 103.0 | 896 | 21.0 | 1371.4 | 0.9 | 4.7 | 104.7 | 1228 |
| | n | 10 | 10 | 10 | 10 | 10 | 10 | 10 | 10 | 10 | 10 | 10 | 10 |
| | SD | 2.1 | 272.6 | 0.6 | 2.7 | 2.7 | 266.6 | 2.1 | 454.9 | 0.7 | 3.7 | 3.7 | 448.8 |
| | SE | 0.7 | 86.2 | 0.2 | 0.9 | 0.9 | 84.3 | 0.7 | 143.9 | 0.2 | 1.2 | 1.2 | 141.9 |
| Curtirsen40 | 1 | | | | | | | | | | | | |
| ip qd*5, | 2 | 18.73 | 495 | 1.0 | 5.6 | 105.6 | 371.9 | 18.40 | 687 | 0.7 | 3.8 | 103.8 | 563.6 |
| twk | 3 | 18.93 | 354 | 1.8 | 10.6 | 110.6 | 229.1 | 18.79 | 392 | 1.7 | 9.8 | 109.8 | 267.5 |
| | 4 | 21.36 | 689 | 0.8 | 3.7 | 103.7 | 550.6 | 21.71 | 817 | 1.1 | 5.4 | 105.4 | 678.0 |
| | 5 | 20.43 | 763 | 0.8 | 3.8 | 103.8 | 621.8 | 20.17 | 923 | 0.5 | 2.5 | 102.5 | 781.6 |
| | 6 | 19.03 | 717 | 0.4 | 2.1 | 102.1 | 571.9 | 19.13 | 1031 | 0.5 | 2.7 | 102.7 | 885.6 |
| | 7 | 20.83 | 1076 | 2.4 | 13.0 | 113.0 | 925.7 | 21.05 | 1324 | 2.6 | 14.2 | 114.2 | 1173.4 |
| | 8 | 20.79 | 483 | 0.8 | 4.2 | 104.2 | 329.0 | 20.78 | 603 | 0.8 | 4.2 | 104.2 | 448.7 |
| | 9 | 18.98 | 593 | 0.6 | 3.2 | 103.2 | 431.2 | 18.47 | 734 | 0.1 | 0.4 | 100.4 | 571.8 |
| | 10 | 19.13 | 671 | 0.2 | 1.2 | 101.2 | 502.7 | 19.87 | 804 | 1.0 | 5.1 | 105.1 | 635.5 |
| | Mean | 19.8 | 649.2 | 1.0 | 5.3 | 105.3 | 503.8 | 19.8 | 812.8 | 1.0 | 5.3 | 105.3 | 667.3 |
| | n | 9 | 9 | 9 | 9 | 9 | 9 | 9 | 9 | 9 | 9 | 9 | 9 |
| | SD | 1.0 | 207.3 | 0.7 | 3.9 | 3.9 | 202.3 | 1.2 | 265.5 | 0.8 | 4.2 | 4.2 | 261.1 |
| | SE | 0.3 | 69.1 | 0.2 | 1.3 | 1.3 | 67.4 | 0.4 | 88.5 | 0.3 | 1.4 | 1.4 | 87.0 |
| | | | | | | % of | Δ | | | | | % of | Δ |
| | | Day 58 | | Δ | Δ | base- | vol- | Day 63 | | Δ | Δ | base- | vol- |
| | | weight | volume | weight | % | line | ume | weight | volume | weight | % | line | ume |
| Vehicle | 1 | 17.5 | 830 | 1.1 | 6.8 | 106.8 | 711.8 | 17.3 | 1255 | 0.9 | 5.8 | 105.8 | 1136.3 |
| i.p. qd*5, | 2 | 22.4 | 1934 | 2.4 | 11.8 | 111.8 | 1809.7 | | | | | | |
| twk | 3 | 23.2 | 2416 | 1.8 | 8.2 | 108.2 | 2283.1 | | | | | | |
| | 4 | 23.8 | 3067 | 1.2 | 5.3 | 105.3 | 2929.9 | | | | | | |
| | 5 | | | | | | | | | | | | |
| | 6 | 22.4 | 1614 | 1.2 | 5.7 | 105.7 | 1469.9 | | | | | | |
| | 7 | 23.2 | 856 | 1.7 | 7.7 | 107.7 | 704.4 | 22.7 | 1007 | 1.1 | 5.3 | 105.3 | 855.2 |
| | 8 | 22.9 | 2006 | 1.8 | 8.6 | 108.6 | 1847.0 | | | | | | |
| | 9 | 20.8 | 2331 | 1.8 | 9.2 | 109.2 | 2169.0 | | | | | | |
| | 10 | | | | | | | | | | | | |
| | Mean | 22.0 | 1881.7 | 1.6 | 7.9 | 107.9 | 1741 | 20.0 | 1130.9 | 1.0 | 5.5 | 105.5 | 996 |
| | n | 8 | 8 | 8 | 8 | 8 | 8 | 2 | 2 | 2 | 2 | 2 | 2 |
| | SD | 2.0 | 768.9 | 0.4 | 2.1 | 2.1 | 766.6 | 3.8 | 175.2 | 0.1 | 0.4 | 0.4 | 198.8 |
| | SE | 0.7 | 271.8 | 0.1 | 0.7 | 0.7 | 271.0 | 2.7 | 123.9 | 0.1 | 0.3 | 0.3 | 140.5 |
| Curtirsen40 | 1 | | | | | | | | | | | | |
| ip qd*5, | 2 | 18.72 | 797 | 1.0 | 5.6 | 105.6 | 673.0 | 18.57 | 1127 | 0.8 | 4.7 | 104.7 | 1003.4 |
| twk | 3 | 18.84 | 560 | 1.7 | 10.0 | 110.0 | 435.6 | 19.25 | 848 | 2.1 | 12.4 | 112.4 | 723.2 |
| | 4 | 22.31 | 1319 | 1.7 | 8.4 | 106.4 | 1180.5 | 22.39 | 2000 | 1.8 | 8.7 | 108.7 | 1861.0 |
| | 5 | 20.77 | 1418 | 1.1 | 5.5 | 105.5 | 1277.2 | 21.04 | 2185 | 1.4 | 6.9 | 106.9 | 2043.7 |
| | 6 | 19.53 | 1693 | 0.9 | 4.8 | 104.8 | 1547.4 | | | | | | |
| | 7 | 21.42 | 1970 | 3.0 | 16.2 | 116.2 | 1819.3 | | | | | | |
| | 8 | 21.45 | 1060 | 1.5 | 7.5 | 107.5 | 905.2 | 22.19 | 1918 | 2.2 | 11.2 | 111.2 | 1763.0 |
| | 9 | 19.51 | 1218 | 1.1 | 6.0 | 106.0 | 1056.5 | 19.81 | 1519 | 1.4 | 7.7 | 107.7 | 1357.1 |
| | 10 | 20.43 | 1475 | 1.5 | 8.1 | 108.1 | 1306.4 | 20.11 | 2426 | 1.2 | 6.4 | 106.4 | 2257.1 |
| | Mean | 20.3 | 1278.9 | 1.5 | 8.0 | 108.0 | 1133.5 | 20.5 | 1717.4 | 1.6 | 8.3 | 108.3 | 1572.6 |
| | n | 9 | 9 | 9 | 9 | 9 | 9 | 7 | 7 | 7 | 1 | 7 | 7 |
| | SD | 1.3 | 434.3 | 0.6 | 3.5 | 3.5 | 426.0 | 1.5 | 575.1 | 0.5 | 2.7 | 2.7 | 563.0 |
| | SE | 0.4 | 144.8 | 0.2 | 1.2 | 1.2 | 142.0 | 0.5 | 217.4 | 0.2 | 1.0 | 1.0 | 212.8 |

### Example 2

### In Vivo Activity of Custirsen (TV-1011) Against RPMI 8226 (Human Myeloma) Xenograft Model, Implanted Subcutaneously into Athymic Nude Mice

A previous in-house study demonstrated that custirsen had an inhibitory effect on tumor growth, but also showed unacceptable toxicity at a high dose.

In this study custirsen is administered using a different dose and regimen in order to avoid toxicity.

### Materials and Methods

### Test Articles

RPMI 8226 (Human Plasmacytoma, Myeloma B Cells) ATCC # CCL-155;
ECM Gel (Matrigel), Sigma-Aldrich, Cat # E1270, 5 ml;
RPMI (Beit Haemek);
Custirsen (TV-1011) 20 mg/ml, K-46138;
Sodium chloride, TEVA.

### Test Animals

120 CB.17 SCID female mice, 4-6 weeks old, 16-20 grams, obtained from Harlan animal breeding center.

### Experimental Procedures

### Cells Preparation

Cells (originated from ATCC) were cultured on RPMI medium. Cell suspension was centrifuged and resuspended in 50% Matrigel/HBSS to a final concentration of 7×10⁷ cells/ml. The suspension was implanted s.c. in the right flank of the anesthetized mouse at a volume of 100 μl.

### Compounds Preparation

11.2 ml of the stock solution of custirsen (TV-1011 20 mg/ml) were added to 44.8 ml saline to receive 4 mg/ml. 28.5 ml of 4 mg/ml were added to 9.5 saline to receive 3 mg/ml.

### Experimental Design

Mice were implanted subcutaneously, with 7×10 RPMI 8226 cells/mouse (in 50% Martigel/HBSS) on Day 0. On day 21, mice were sorted by the optimal average tumor volume (˜130 mm³) and were allocated into 2 groups of 10 mice each.

| Gr. | N | Agent | Route | Dose & schedule |
|---|---|---|---|---|
| 1 | 10 | Vehicle | ip | qd*5, then biwk |
| 2 | 10 | Custirsen | ip | 30 qd*5, 40 biwk |

“qd*5” means daily dosing for 5 days;
“biwk” means twice per week;
“twk” means three times per week.

The treatment started on day 21 post implant, 0.2 ml per 20 grams v/w.

### Statistical Analysis

Tumor volume was calculated as follows:

$$π \times {( \frac{width}{2} )}^{2} \times length .$$

The analysis of weight gain and tumor volume progression was made using one-way ANOVA followed by Tukey post-hoc comparisons.

### Results

The treatment responses for Day 62 are summarized in FIG. 2 and Table 5.

Treatment with custirsen inhibited tumor growth compared to control group (Table 3). Custirsen alone inhibited tumor growth by 67% (p<0.001).

TABLE 4
Summary of the results (day 62)

| | | | Maintenance | | | | | | | | |
|---|---|---|---|---|---|---|---|---|---|---|---|
| | | Loading | Dose, mg/ | Total | Δ mean | | | | Max BW | | |
| | | Dose, mg/ | kg/week | dose, | tumor volume, | | No. of | No. of | (mean) | No. of | No. og |
| Compound | Regimen | kg/week | *5 w | mg/kg | (mean ± se) | % TGI | PR | CR | reduction | TRD | nTRD |
| Vehicle | i.p. qd*5, | | | | 1746 ± 218 | | 0 | 0 | −1.7% | 0 | 0 |
| | then biwk | | | | | | | | day 28 | | |
| Custirsen | i.p. qd*5, | 150 | 80 * 5 | 550 | 574 ± 118 | 67*** | 1 | 0 | −0.5% | 0 | 0 |
| | then biwk | | | | | | | | day 28 | | |

*p < 0.05,
** p < 0.01,
***p < 0.001 (one-way Anova, Tukey post-hoc test)

### Conclusions

Custirsen 30 mg/kg qd×5 then 40 mg/kg biwk—alone was very efficacious; this regimen was not toxic;
The effect of custirsen on tumor volume was statistically significant. See Table 4.

TABLE 5
Raw Data for Custirsen Monotherapy for Treatment of Myeloma

| | | | | | | | | % of | | | | | % of |
|---|---|---|---|---|---|---|---|---|---|---|---|---|---|
| | | Day 21 | | Day 28, | | Δ | Δ | base- | Day 33, | | Δ | Δ | base- |
| | | weight | volume | weight | volume | weight | % | line | weight | volume | weight | % | line |
| Vehicle | 1 | 17.8 | 100 | 17.0 | 129 | −0.8 | −4.3 | 95.7 | 17.0 | 185 | −0.8 | −4.6 | 95.4 |
| i.p. qd*5, | 2 | 19.2 | 113 | 19.5 | 150 | 0.3 | 1.7 | 101.7 | 20.6 | 253 | 1.4 | 7.4 | 107.4 |
| biwk | 3 | 21.1 | 116 | 21.6 | 244 | 0.4 | 2.1 | 102.1 | 21.1 | 270 | 0.0 | −0.2 | 99.8 |
| | 4 | 18.7 | 119 | 18.3 | 209 | −0.4 | −2.4 | 97.6 | 19.1 | 189 | 0.3 | 1.7 | 101.7 |
| | 5 | 18.1 | 124 | 17.0 | 199 | −1.1 | −6.1 | 93.9 | 17.4 | 274 | −0.6 | −3.8 | 96.4 |
| | 6 | 21.5 | 126 | 20.7 | 260 | −0.8 | −3.7 | 96.3 | 20.4 | 357 | −1.1 | −5.1 | 94.9 |
| | 7 | 20.3 | 132 | 20.1 | 183 | −0.2 | −0.9 | 99.1 | 20.0 | 171 | −0.2 | −1.2 | 98.8 |
| | 8 | 18.7 | 141 | 18.8 | 224 | 0.1 | 0.5 | 100.5 | 18.8 | 279 | 0.1 | 0.4 | 100.4 |
| | 9 | 19.1 | 154 | 19.1 | 277 | 0.0 | 0.1 | 100.1 | 18.9 | 334 | −0.2 | −1.2 | 96.8 |
| | 10 | 21.6 | 157 | 21.0 | 215 | −0.8 | −3.6 | 96.4 | 20.5 | 176 | −1.3 | −5.9 | 94.1 |
| | Mean | 19.6 | 128.5 | 19.3 | 214.1 | −0.3 | −1.7 | 96.3 | 19.4 | 246.9 | −0.3 | −1.2 | 98.8 |
| | n | 10 | 10 | 10 | 10 | 10 | 10 | 10 | 10 | 10 | 10 | 10 | 10 |
| | SD | 1.5 | 18.2 | 1.6 | 45.9 | 0.5 | 2.8 | 2.6 | 1.4 | 68.9 | 0.8 | 3.9 | 3.9 |
| | SE | 0.5 | 5.7 | 0.5 | 14.5 | 0.2 | 0.9 | 0.9 | 0.4 | 21.8 | 0.2 | 1.2 | 1.2 |
| Curtirsen | 1 | 21.4 | 107 | 21.0 | 78 | −0.4 | −2.0 | 98.0 | 20.9 | 132 | −0.5 | −2.2 | 97.8 |
| 30 mg/kg | 2 | 17.7 | 113 | 16.5 | 127 | 0.8 | 4.4 | 104.4 | 19.2 | 131 | 1.5 | 8.3 | 108.3 |
| qd*5, | 3 | 20.5 | 116 | 21.3 | 114 | 0.8 | 4.0 | 104.0 | 22.1 | 120 | 1.7 | 8.1 | 106.1 |
| 40 mg/kg | 4 | 15.9 | 119 | 16.3 | 149 | 0.4 | 2.6 | 102.6 | 15.2 | 116 | −0.6 | −4.1 | 95.9 |
| biwk | 5 | 19.6 | 123 | 16.7 | [symbol] | −0.9 | −4.8 | 95.2 | 18.4 | 167 | −1.2 | −6.0 | 94.0 |
| | 6 | 20.3 | [symbol] | 19.6 | 204 | −0.6 | −3.1 | 96.9 | 20.5 | 157 | 0.3 | 1.3 | 101.3 |
| | 7 | 17.9 | 131 | 16.9 | 159 | −1.0 | −5.6 | 94.4 | 17.6 | 100 | −0.3 | −1.7 | 96.3 |
| | 8 | 16.6 | [symbol] | 17.1 | 175 | 0.5 | 3.0 | 103.0 | 17.5 | 154 | 0.9 | 5.5 | 105.5 |
| | 9 | 20.1 | 147 | 19.9 | 193 | −0.3 | −1.3 | 98.7 | 20.1 | 206 | 0.0 | −0.1 | 99.9 |
| | 10 | 17.8 | 172 | 17.5 | 204 | −0.3 | −1.7 | 98.3 | 19.1 | 186 | 1.3 | 7.3 | 107.3 |
| | Mean | 18.8 | 129.3 | 18.7 | 158.1 | −0.1 | −0.5 | 99.5 | 19.1 | 147.0 | 0.3 | 1.6 | 101.6 |
| | n | 10 | 10 | 10 | 10 | 10 | 10 | 10 | 10 | 10 | 10 | 10 | 10 |
| | SD | 1.8 | 19.2 | 1.7 | 41.7 | 0.7 | 3.7 | 3.7 | 2.0 | 33.3 | 1.0 | 5.3 | 5.3 |
| | SE | 0.6 | 6.1 | 0.6 | 13.2 | 0.2 | 1.2 | 1.2 | 0.6 | 10.5 | 0.3 | 1.7 | 1.7 |

| | | | | | | % of | | | | | % of |
|---|---|---|---|---|---|---|---|---|---|---|---|
| | | Day 41, | | Δ | Δ | base- | Day 48, | | Δ | Δ | base- |
| | | weight | volume | weight | % | line | weight | volume | weight | % | line |
| Vehicle | 1 | 16.9 | 235 | −0.8 | −4.6 | 95.4 | 16.8 | 341 | −1.0 | −5.5 | 94.5 |
| i.p. qd*5, | 2 | 20.0 | 359 | 0.8 | 4.1 | 104.1 | 21.7 | 514 | 2.6 | 13.4 | 113.4 |
| biwk | 3 | 21.1 | 368 | 0.0 | 0.1 | 100.1 | 21.6 | 460 | 0.5 | 2.2 | 102.2 |
| | 4 | 18.9 | 285 | 0.2 | 0.9 | 100.9 | 19.5 | 674 | 0.7 | 4.0 | 104.0 |
| | 5 | 18.1 | 336 | 0.1 | 0.3 | 100.3 | 18.2 | 384 | 0.1 | 0.6 | 100.6 |
| | 6 | 21.6 | 509 | 0.1 | 0.3 | 100.3 | 22.3 | 813 | 0.8 | 3.6 | 103.6 |
| | 7 | 20.3 | 481 | 0.1 | 0.3 | 100.3 | 20.5 | 726 | 0.3 | 1.3 | 101.3 |
| | 8 | 20.2 | 449 | 1.5 | 7.8 | 107.8 | 19.8 | 755 | 0.8 | 4.5 | 104.5 |
| | 9 | 19.4 | 607 | 0.3 | 1.5 | 101.5 | 20.1 | 1104 | 1.1 | 5.5 | 105.5 |
| | 10 | 20.6 | 264 | −1.2 | −5.6 | 94.4 | 21.2 | 410 | −0.6 | −2.9 | 97.1 |
| | Mean | 19.7 | 389.5 | 0.1 | 0.5 | 100.5 | 20.1 | 618.0 | 0.5 | 2.7 | 102.7 |
| | n | 10 | 10 | 10 | 10 | 10 | 10 | 10 | 10 | 10 | 10 |
| | SD | 1.4 | 119.4 | 0.7 | 3.8 | 3.8 | 1.7 | 240.0 | 1.0 | 5.1 | 5.1 |
| | SE | 0.4 | 37.7 | 0.2 | 1.2 | 1.2 | 0.5 | 75.9 | 0.3 | 1.6 | 1.6 |
| Curtirsen | 1 | 21.7 | 76 | 0.3 | 1.4 | 101.4 | 23.2 | 101 | 1.8 | 8.5 | 108.5 |
| 30 mg/kg | 2 | 20.0 | 144 | 2.3 | 12.9 | 112.9 | 20.6 | 360 | 3.0 | 16.7 | 116.7 |
| qd*5, | 3 | 22.8 | 140 | 2.3 | 11.2 | 111.2 | 23.1 | 268 | 2.6 | 12.9 | 112.9 |
| 40 mg/kg | 4 | 17.2 | 139 | 1.3 | 8.4 | 108.4 | 19.1 | 267 | 3.3 | 20.7 | 120.7 |
| biwk | 5 | 20.2 | 336 | 0.6 | 2.9 | 102.9 | 21.6 | 556 | 2.0 | 10.1 | 110.1 |
| | 6 | 20.7 | 249 | 0.5 | 2.3 | 102.3 | 21.6 | 581 | 1.5 | 7.6 | 107.6 |
| | 7 | 18.9 | 192 | 1.1 | 5.9 | 105.9 | 20.5 | 211 | 2.6 | 14.4 | 114.4 |
| | 8 | 18.7 | 185 | 2.0 | 12.3 | 112.3 | 20.5 | 257 | 3.9 | 23.3 | 123.3 |
| | 9 | 21.5 | 223 | 1.3 | 6.7 | 106.7 | 23.3 | 451 | 3.2 | 15.7 | 115.7 |
| | 10 | 18.5 | 200 | 0.7 | 3.9 | 103.9 | 19.3 | 392 | 1.5 | 8.4 | 108.4 |
| | Mean | 20.0 | 184.3 | 1.2 | 6.8 | 106.8 | 21.3 | 344.4 | 2.5 | 13.8 | 113.8 |
| | n | 10 | 10 | 10 | 10 | 10 | 10 | 10 | 10 | 10 | 10 |
| | SD | 1.7 | 72.4 | 0.8 | 4.3 | 4.3 | 1.6 | 153.2 | 0.8 | 5.4 | 5.4 |
| | SE | 0.5 | 22.9 | 0.2 | 1.3 | 1.3 | 0.5 | 48.4 | 0.3 | 1.7 | 1.7 |

| | | | | | | % of | | | | | % of | Δ |
|---|---|---|---|---|---|---|---|---|---|---|---|---|
| | | Day 55 | | Δ | Δ | base- | Day 62 | | Δ | Δ | base- | vol- |
| | | weight | volume | weight | % | line | weight | volume | weight | % | line | ume |
| Vehicle | 1 | 17.4 | 540 | −0.4 | −2.3 | 97.7 | 17.8 | 1005 | 0.0 | 0.3 | 100.3 | 905.5 |
| i.p. qd*5, | 2 | 21.8 | 1417 | 2.6 | 13.7 | 113.7 | 22.9 | 2026 | 3.7 | 19.3 | 119.3 | 1913.4 |
| biwk | 3 | 22.0 | 900 | 0.8 | 4.0 | 104.0 | 22.2 | 1554 | 1.1 | 5.0 | 105.0 | 1437.1 |
| | 4 | 21.4 | 1242 | 2.6 | 14.1 | 114.1 | 21.1 | 2256 | 2.4 | 12.7 | 112.7 | 2137.4 |
| | 5 | 19.6 | 544 | 1.6 | 8.6 | 108.6 | 19.0 | 953 | 0.9 | 4.8 | 104.8 | 829.0 |
| | 6 | 23.0 | 1495 | 1.5 | 6.9 | 106.9 | 23.7 | 2418 | 2.2 | 10.0 | 110.0 | 2289.3 |
| | 7 | 22.0 | 1100 | 1.7 | 8.4 | 108.4 | 22.1 | 2252 | 1.8 | 8.9 | 108.9 | 2119.3 |
| | 8 | 21.1 | 1260 | 2.4 | 12.7 | 112.7 | 22.2 | 2291 | 3.5 | 18.8 | 118.8 | 2150.2 |
| | 9 | 20.9 | 1667 | 1.8 | 9.3 | 109.3 | 22.4 | 2945 | 3.3 | 17.1 | 117.1 | 2790.8 |
| | 10 | 22.3 | 599 | 0.5 | 2.2 | 102.2 | 23.0 | 1040 | 1.2 | 5.4 | 105.4 | 882.7 |
| | Mean | 21.1 | 1076.5 | 1.5 | 7.8 | 107.8 | 21.6 | 1874.0 | 2.0 | 10.2 | 110.2 | 1745.6 |
| | n | 10 | 10 | 10 | 10 | 10 | 10 | 10 | 10 | 10 | 10 | 10 |
| | SD | 1.6 | 412.5 | 1.0 | 5.3 | 5.3 | 1.9 | 693.6 | 1.2 | 6.6 | 6.6 | 687.9 |
| | SE | 0.5 | 130.4 | 0.3 | 1.7 | 1.7 | 0.6 | 219.3 | 0.4 | 2.1 | 2.1 | 217.5 |
| Curtirsen | 1 | 22.1 | 106 | 0.7 | 3.3 | 103.3 | 22.7 | 92 | 1.3 | 5.9 | 105.9 | −14.3 |
| 30 mg/kg | 2 | 20.7 | 638 | 3.0 | 16.7 | 116.7 | 21.5 | 955 | 3.9 | 21.8 | 121.8 | 842.5 |
| qd*5, | 3 | 22.4 | 463 | 2.0 | 9.7 | 109.7 | 22.8 | 891 | 2.3 | 11.4 | 111.4 | 775.4 |
| 40 mg/kg | 4 | 19.5 | 399 | 3.6 | 22.9 | 122.9 | 18.8 | 568 | 3.0 | 18.7 | 118.7 | 448.8 |
| biwk | 5 | 20.2 | 767 | 0.6 | 3.1 | 103.1 | 20.8 | 1324 | 1.2 | 6.2 | 106.2 | 1200.9 |
| | 6 | 21.1 | 663 | 0.8 | 4.0 | 104.0 | 21.7 | 804 | 1.4 | 7.1 | 107.1 | 676.2 |
| | 7 | 19.0 | 257 | 1.1 | 6.1 | 106.1 | 19.1 | 379 | 1.2 | 6.8 | 105.8 | 247.2 |
| | 8 | 19.0 | 246 | 2.3 | 14.0 | 114.0 | 19.8 | 273 | 3.2 | 19.0 | 119.0 | 135.4 |
| | 9 | 21.8 | 638 | 1.7 | 8.4 | 108.4 | 22.6 | 1001 | 2.5 | 12.4 | 112.4 | 853.6 |
| | 10 | 18.4 | 588 | 0.7 | 3.7 | 103.7 | 18.9 | 742 | 1.1 | 6.3 | 106.3 | 570.4 |
| | Mean | 20.4 | 476.6 | 1.7 | 9.2 | 109.2 | 20.9 | 702.9 | 2.1 | 11.5 | 111.5 | 573.6 |
| | n | 10 | 10 | 10 | 10 | 10 | 10 | 10 | 10 | 10 | 10 | 10 |
| | SD | 1.4 | 218.0 | 1.1 | 6.7 | 6.7 | 1.6 | 375.1 | 1.0 | 6.2 | 6.2 | 373.4 |
| | SE | 0.5 | 68.9 | 0.3 | 2.1 | 2.1 | 0.5 | 118.6 | 0.3 | 1.9 | 1.9 | 118.1 |

[symbol]

indicates data missing or illegible when filed

### Example 3

### In Vivo Activity of Custirsen (OGX-11) Against PC-3 (Human Prostate Carcinoma) Xenograft Model, Implanted Subcutaneously into Athymic Nude Mice

The cells were implanted subcutaneously into female immunodeficient nude mice. On day 14, when the tumors reached 90-135=³, mice were sorted into treatment groups (n=10): 1. Control group was treated with saline i.p. qd*7+twk; 2. Custirsen treatment (25 mg/kg ip qd*5+twk).

Tumors and body weights were measured once a week until termination of the study on day 58 included. Response to treatment was evaluated for tumor growth inhibition (TGI) and expressed as the difference between the mean tumor volumes of treated and control mice.

### Materials and Methods

a. Materials
PC-3 (Human Prostate Adenocarcinoma), ATCC, CAL-1435™
RPMI medium 1640+L-Glutamine (Beit Haemek)
Custirsen (OGX) 20 mg/ml, K-46138;
The custirsen solution was prepared once weekly and stored at 4° C. 4.0 ml of the stock solution of custirsen (20 mg/ml) were added to 60 ml saline. (1:16 dilution)
Saline (TEVA).
   b. Animals

Mutant Athymic Nude female mice, 4-6 weeks old, 16-20 grams, obtained from Harlan animal breeding center.

c. Cell Preparation

Harvested 20 flasks (T-175), passage 7, which were split 1:4. Cells were cultured (P-3, originated from ATCC). A sample from the cell suspension was taken for counting (0.3 ml in duplicates for CEDEX) before spun down. Cell viability was 99.4% and live cell concentration was 49.2×10 cells/ml and pellet was re-suspended with HESS to a final concentration of 3×10⁷/ml (3×10⁶ cells/0.1 ml/mouse).

d. Study Design

Tumors were implanted subcutaneously with PC-3 cells in the right dorsal of the mouse on Day 0. Each mouse was injected with 0.1 ml cell suspension from a concentration of 3×10⁷ cells/ml. On day 14, mice were sorted by the optimal tumor volume (90-135 mm³) and were allocated into groups of 10 mice (Table 6). Mice were individually tagged and their tumor volume and body weight were monitored weekly during the study. Tumor size was measured by caliper and calculated using the formula:

$$π \times {( \frac{width}{2} )}^{2} \times length .$$

The treatment started on day 15 (0.2 ml per 20 grams v/w) and continued till day 58. Treatment regimen and doses are indicated in Table 6.

### Results

The treatment responses are summarized in Table 7 and presented in FIG. 3.

The custirsen (OGX) treatment at 25 mg/kg alone had a moderate effect by itself, 32% TGI (not significant) compare to the vehicle group. Additionally, the stand alone efficacy of custirsen at 25 mg/kg was significantly different than that of vehicle at 1 time point (4 weeks).

TABLE 6
Experimental design

Drug and treatment

| Gr. | N | Agent | mg/kg | Route | Schedule |
|---|---|---|---|---|---|
| 1 | 10 | Vehicle | — | ip | qd*5 + twk |
| 2 | 10 | Custrisen | 25 | ip | qd*5 + twk |

ip—intraperitoneal;
qd—every day;
twk—three times a week;
qd*5—daily dosing for 5 days.

TABLE 7
Summary of the results (day 58)
Antitumor Activity of Custirsen (OGX-11; TV-1011) against PC-3
Started 14 days post implant
Ended 58 Days post implant

| | | | | Tumor | | | | |
|---|---|---|---|---|---|---|---|---|
| Drugs | | | | Delta Mean | | | Animals | |
| | | | Dose | Tumor Volume | | | Delta % | Dead/ |
| Compount | Route | Regimen | (mg/kg) | (mm3 ± SEM) | % T/C | % TGI | Body Wt. | Total |
| Saline | i.p | qd*5 + twk | — | 569 ± 79.48 | — | | 1.4 ± 1.29 | 0/10 |
| OGX | i.p | qd*5 + twk | 25 mg/kg | 385 ± 93.95 | 68 | 32 | 7.61 ± 2.08 | 0/10 |

* p < 0.05,
** p < 0.01,
*** p < 0.001 (one-way Anova, Tukey post-hoc test)

### Conclusion

Although custirsen is not currently a therapeutic agent as a stand alone standard care, it demonstrated in this study a moderate effect of 32% TGI (not statistically significant) at a higher tested dose (than pervious studies) of 25 mg/kg. The effect of custirsen monotherapy was statistically significant at the week 4 time point, however.

TABLE 8
Raw Data. Tumor volumes and body weights

| Tumor Volume | Day: | 14 | 22 | DELTA | 29 | DELTA | 38 | DELTA | 43 | DELTA | 51 | DELTA | 58 | DELTA |
|---|---|---|---|---|---|---|---|---|---|---|---|---|---|---|
| Group 1 | 1 | 96 | 227 | 131 | 259 | 163 | 422 | 326 | 484 | 389 | 362 | 256 | 394 | 298 |
| Vehicle | 2 | 99 | 308 | 209 | 386 | 287 | 624 | 525 | 737 | 639 | 859 | 760 | 1057 | 958 |
| Saline | 3 | 102 | 198 | 96 | 333 | 231 | 393 | 291 | 478 | 376 | 518 | 416 | 612 | 510 |
| qd*5 + twk | 4 | 106 | 175 | 69 | 289 | 182 | 412 | 305 | 444 | 338 | 537 | 431 | 636 | 529 |
| | 5 | 107 | 189 | 81 | 435 | 327 | 536 | 429 | 623 | 516 | 738 | 631 | 770 | 662 |
| | 6 | 113 | 172 | 59 | 258 | 144 | 408 | 294 | 419 | 305 | 388 | 275 | 513 | 400 |
| | 7 | 116 | 271 | 156 | 425 | 309 | 567 | 451 | 742 | 626 | 763 | 648 | 1119 | 1003 |
| | 8 | 119 | 235 | 117 | 317 | 199 | 415 | 296 | 482 | 364 | 457 | 338 | 476 | 357 |
| | 9 | 120 | 214 | 94 | 430 | 310 | 459 | 339 | 571 | 451 | 513 | 393 | 767 | 647 |
| | 10 | 126 | 246 | 120 | 382 | 256 | 628 | 502 | 737 | 611 | 705 | 580 | 446 | 321 |
| Average | | 110 | 224 | 113 | 351 | 241 | 486 | 376 | 572 | 461 | 584 | 474 | 679 | 569 |
| STD Dev | | 9.82 | 43.44 | 44.77 | 69.45 | 66.63 | 93.32 | 91.72 | 129.26 | 127.32 | 170.44 | 170.21 | 250.13 | 251.33 |
| SEM | | 3.10 | 13.74 | 14.16 | 21.96 | 21.07 | 29.51 | 29.01 | 40.88 | 40.26 | 53.90 | 53.82 | 79.10 | 79.48 |
| Group 2 | 1 | 98 | 129 | 32 | 147 | 50 | 124 | 27 | 98 | 0 | 0 | −98 | 0 | −98 |
| Custirsen | 2 | 98 | 109 | 11 | 134 | 36 | 200 | 102 | 302 | 204 | 278 | 180 | 344 | 246 |
| qd*5 + twk | 3 | 103 | 177 | 74 | 221 | 118 | 372 | 269 | 401 | 298 | 433 | 330 | 526 | 423 |
| 25 mg/kg | 4 | 105 | 179 | 74 | 261 | 156 | 519 | 413 | 452 | 346 | 612 | 507 | 128 | 23 |
| | 5 | 110 | 213 | 102 | 246 | 136 | 278 | 168 | 192 | 82 | 135 | 24 | 807 | 697 |
| | 6 | 113 | 258 | 145 | 356 | 244 | 625 | 512 | 708 | 596 | 717 | 605 | 895 | 782 |
| | 7 | 114 | 223 | 109 | 285 | 171 | 366 | 252 | 498 | 384 | 599 | 485 | 698 | 584 |
| | 8 | 118 | 204 | 587 | 236 | 119 | 321 | 203 | 473 | 355 | 377 | 259 | 259 | 142 |
| | 9 | 122 | 204 | 82 | 323 | 202 | 409 | 287 | 475 | 354 | 570 | 448 | 558 | 437 |
| | 10 | 124 | 200 | 75 | 240 | 116 | 444 | 319 | 481 | 356 | 596 | 472 | 740 | 615 |
| Average | | 110 | 190 | 79 | 245 | 135 | 366 | 255 | 408 | 298 | 432 | 321 | 496 | 385 |
| STD Dev | | 9.38 | 43.76 | 37.75 | 69.09 | 63.24 | 146.75 | 142.31 | 172.77 | 167.51 | 233.17 | 227.92 | 302.09 | 297.08 |
| SEM | | 2.97 | 13.84 | 11.94 | 21.85 | 20.00 | 46.41 | 45.00 | 54.63 | 52.97 | 73.73 | 72.07 | 95.53 | 93.95 |

| Body Weight | Day: | 14 | 22 | Δ (%) | 29 | Δ (%) | 38 | Δ (%) | 43 | Δ (%) | 51 | Δ (%) | 58 | Δ (%) |
|---|---|---|---|---|---|---|---|---|---|---|---|---|---|---|
| Group 1 | 1 | 23.75 | 23.91 | 0.68 | 23.62 | −0.57 | 24.38 | 2.62 | 24.14 | 1.62 | 23.04 | −3.00 | 23.08 | −2.83 |
| Vehicle | 2 | 22.28 | 22.80 | 2.37 | 22.82 | 2.44 | 23.26 | 4.43 | 23.25 | 4.38 | 23.74 | 6.57 | 23.29 | 4.55 |
| Saline | 3 | 22.89 | 23.39 | 3.08 | 23.23 | 2.40 | 23.09 | 1.79 | 23.09 | 1.76 | 23.20 | 2.26 | 23.07 | 1.69 |
| qd*5 + twk | 4 | 21.70 | 21.14 | −2.56 | 21.79 | 0.43 | 22.17 | 2.17 | 22.44 | 3.41 | 22.86 | 5.35 | 22.56 | 3.97 |
| | 5 | 24.41 | 24.93 | 2.09 | 25.20 | 3.23 | 24.44 | 0.09 | 23.95 | −1.92 | 24.12 | −1.20 | 23.33 | −4.44 |
| | 6 | 24.00 | 25.14 | 4.74 | 25.37 | 5.70 | 25.77 | 7.39 | 26.06 | 8.56 | 26.66 | 11.08 | 25.66 | 6.92 |
| | 7 | 23.28 | 23.58 | 1.27 | 24.48 | 5.13 | 24.03 | 3.21 | 24.01 | 3.13 | 24.91 | 6.99 | 24.26 | 4.20 |
| | 8 | 22.22 | 22.03 | −0.86 | 23.06 | 3.79 | 23.33 | 5.01 | 23.56 | 6.03 | 23.04 | 3.70 | 23.22 | 4.51 |
| | 9 | 25.60 | 24.94 | −2.58 | 26.54 | 3.67 | 26.15 | 2.16 | 26.13 | 2.06 | 25.40 | −0.77 | 24.55 | −4.09 |
| | 10 | 24.54 | 23.94 | −2.43 | 25.28 | 3.02 | 24.57 | 0.15 | 24.78 | 1.01 | 25.28 | 3.03 | 24.43 | −0.43 |
| Average | | 23.45 | 23.58 | 0.58 | 24.14 | 2.93 | 24.12 | 2.90 | 24.14 | 3.00 | 24.23 | 3.40 | 23.75 | 1.40 |
| STD Dev | | 1.23 | 1.30 | 2.59 | 1.46 | 1.91 | 1.22 | 2.23 | 1.21 | 2.88 | 1.28 | 4.31 | 0.94 | 4.08 |
| SEM | | 0.39 | 0.41 | 0.82 | 0.46 | 0.60 | 0.39 | 0.71 | 0.38 | 0.91 | 0.41 | 1.36 | 0.30 | 1.29 |
| Group 2 | 1 | 22.55 | 24.36 | 8.05 | 23.12 | 2.54 | 23.86 | 5.83 | 25.10 | 11.31 | 25.06 | 11.15 | 25.19 | 11.72 |
| Custirsen | 2 | 22.84 | 24.77 | 8.43 | 23.82 | 4.27 | 25.07 | 9.77 | 26.82 | 17.42 | 25.95 | 13.61 | 25.61 | 12.12 |
| qd*5 + twk | 3 | 21.68 | 23.33 | 7.61 | 19.88 | −8.31 | 23.16 | 6.81 | 24.75 | 14.15 | 23.36 | 7.74 | 23.50 | 8.39 |
| 25 mg/kg | 4 | 23.27 | 23.78 | 2.23 | 19.96 | −14.20 | 22.57 | −2.97 | 24.98 | 7.35 | 22.85 | −1.78 | 26.73 | 14.89 |
| | 5 | 23.06 | 22.95 | −0.47 | 23.59 | 2.28 | 25.65 | 11.23 | 26.26 | 13.87 | 26.86 | 16.47 | 22.28 | −3.39 |
| | 6 | 22.35 | 22.85 | 2.27 | 21.29 | −4.72 | 22.14 | −0.93 | 23.30 | 4.28 | 22.77 | 1.90 | 22.29 | −0.25 |
| | 7 | 24.33 | 26.74 | 9.92 | 25.50 | 4.83 | 25.93 | 6.59 | 26.92 | 10.65 | 26.31 | 8.14 | 26.55 | 9.12 |
| | 8 | 23.49 | 27.32 | 16.30 | 25.41 | 8.17 | 26.52 | 12.91 | 28.16 | 19.87 | 27.91 | 18.83 | 27.31 | 16.28 |
| | 9 | 21.68 | 24.75 | 14.17 | 23.07 | 6.43 | 22.88 | 5.54 | 23.38 | 7.87 | 23.95 | 10.50 | 22.83 | 5.33 |
| | 10 | 20.35 | 21.37 | 4.99 | 21.22 | 4.25 | 21.44 | 5.35 | 21.79 | 7.06 | 20.84 | 2.40 | 20.74 | 1.91 |
| Average | | 22.56 | 24.22 | 7.35 | 22.68 | 0.55 | 23.92 | 6.01 | 25.14 | 11.38 | 24.59 | 8.90 | 24.30 | 7.61 |
| STD Dev | | 1.12 | 1.80 | 5.31 | 2.03 | 7.22 | 1.76 | 4.94 | 1.95 | 4.95 | 2.20 | 6.61 | 2.26 | 6.57 |
| SEM | | 0.35 | 0.57 | 1.68 | 0.64 | 2.28 | 0.56 | 1.56 | 0.62 | 1.57 | 0.70 | 2.09 | 0.72 | 2.08 |

Secretary clusterin (sCLU)-2 is a stress-activated, cytoprotective chaperone that confers broad-spectrum cancer treatment resistance and its targeted inhibitor (TV-1011) is currently in Phase III trials for prostate cancer. TV-1011, also known as custirsen, which can be in the form of custirsen sodium, inhibits the production of clusterin, a protein that is associated with treatment resistance in a number of solid tumors and hematological cancer, including human myeloma (plasmacytoma, B cells) along with prostate, breast, non-small cell lung, ovarian, and bladder cancers. It has potential applicability as a therapeutic in a broad number of cancers at different stages and can potentially be used in combination with a variety of commonly used cancer treatments, including chemotherapy, radiation therapy, and hormone ablation therapy.

The present invention relates to the surprising discovery that custirsen is effective for cancer treatment as a monotherapy.

### REFERENCES

1. D'Addario et al., Metastatic non-small-cell lung cancer: ESMO Clinical Practice Guidelines for diagnosis, treatment and follow-up. Annals of Oncology.
2. Fidias and Novello, Strategies for Prolonged Therapy in Patients with Advanced Non-Small-Cell Lung Cancer. Journal of Clinical Oncology, 2010, 28(34): 5116-5123.
3. Jamal et al., Global Cancer statistics, 2011. CA Cancer J Clin; 2011, 61:69-90.
4. Langer et al., The Evolving Role of Histology in the Management of Advanced Non-Small-Cell Lung Cancer. Journal of Clinical Oncology, 2010, 28(36):5311-5320.
5. National Comprehensive Cancer Network Clinical Practice Guidelines in Oncology, Non-Small Cell Lung Cancer, V.2.2010. National Comprehensive Cancer Network, Inc., Mar. 5, 2010.
6. National Cancer Institute, General Information about Non-Small Cell Lung Cancer. http://www.cancer.gov/CANCERTOPICS/PDWTREATMENT/NON-SMALL-CELL-LUNG/PATIENT, accessed Feb. 24, 2011.

1. A method of treating cancer in a subject afflicted with cancer comprising administering to the subject an anti-clusterin oligonucleotide as a monotherapy to treat the cancer.

2. The method of claim 1, wherein the anti-clusterin oligonucleotide is administered to the subject periodically.

3. The method of claim 1, wherein the anti-clusterin oligonucleotide comprises nucleotides in the sequence CAGCAGCAGAGTCTTCATCAT (Seq. ID No.: 1).

4. The method of claim 1, wherein the anti-clusterin oligonucleotide is modified to increase its stability in vivo.

5. The method of claim 4, wherein the anti-clusterin oligonucleotide has a phosphorothioate backbone throughout, has sugar moieties of nucleotides 1-4 and 18-21 bearing 2′-O-methoxyethyl modifications, has nucleotides 5-17 which are 2′deoxynucleotides, and has 5-methylcytosines at nucleotides 1, 4, and 19.

6. The method of claim 1, wherein the patient is afflicted with myeloma.

7. The method of claim 1, wherein the patient is afflicted with prostate cancer.

8. The method of claim 1, wherein the cancer is unresectable, advanced or metastatic cancer.

9. The method of claim 1, wherein the subject is a mammalian subject.

10. The method of claim 1, wherein the mammalian subject is a human subject.

11. The method of claim 1, wherein the anti-clusterin oligonucleotide is administered to the subject intravenously in an aqueous solution comprising sodium ions.

12. The method of claim 1, wherein the anti-clusterin oligonucleotide is administered to the subject as 3 separate loading doses within a 5 to 9 day period at the beginning of treatment and then once weekly thereafter.

13. The method of claim 12, wherein the dose of the anti-clusterin oligonucleotide increases over each of the 3 loading doses.

14. The method of claim 12, wherein the first, second, and third loading doses are 320, 480, and 640 mg, respectively.

15. The method of claim 10, wherein 640 mg of the anti-clusterin oligonucleotide is administered to the human subject.

16. A composition for treating cancer in a subject afflicted with cancer, comprising an anti-clusterin oligonucleotide having the sequence CAGCAGCAGAGTCTTCATCAT (Seq. ID No.: 1).

17-20. (canceled)

21. A package for use in the treatment cancer in a subject afflicted with cancer, comprising an anti-clusterin oligonucleotide having the sequence CAGCAGCAGAGTCTTCATCAT (Seq. ID No.: 1).


SEQUENCE LISTING (1 sequence)

SEQ ID NO: 1 - DNA, 21 bp, Homo sapiens
cagcagcagagtcttcatcat